(19) United States
(12) Patent Application Publication
(10) Pub. No.: US 2011/0186054 A1
(43) Pub. Date: Aug. 4, 2011

(54) Apparatus for Facilitating Respiration During Nasal Congestion, and Related Methods

(76) Inventor: Bryan Boyd, San Diego, CA (US)

(21) Appl. No.: 12/698,080

(22) Filed: Feb. 1, 2010

(51) Int. Cl. A61F 5/37

(52) U.S. Cl. 128/846

(57) ABSTRACT

Disclosed are an apparatus and related methods for facilitating nasal congested respiration.

### CROSS-REFERENCE TO RELATED APPLICATIONS

N/A

### STATEMENT REGARDING FEDERALLY SPONSORED RESEARCH OR DEVELOPMENT

N/A

### BACKGROUND OF THE INVENTION

1. Field of Invention

The present invention is in the field of apparatus and methods for facilitating respiration during nasal congestion.

2. Background of the Invention

Conditions are known to arise wherein a human's nasal cavity becomes congested. For example, after an operation to remedy a deviated septum, bandaging and/or medication may congest or block the recovering patient's nasal cavity. Other conditions exist and are well known, although not specifically recited, wherein a human's nasal cavity may become congested. Nasal congestion or blockage can become problematic since the nasal cavity is a primary intake for respiration, particularly during slumber, and blockage thereof can cause discomfort, e.g., breathing difficulty or sleep apnea.

The above mentioned problem is particularly exacerbated under circumstances wherein the oral cavity (throat-and-mouth cavity), the other primary intake for respiration, is potentially obstructed because respiration may be inadvertently cut-off. For example, a human with a congested nasal cavity may unconsciously seal the throat-and-mouth cavity whenever the human's tongue contacts the palate (i.e., roof of the human's mouth) whereby the human's breathing is discontinued. The identified condition is further problematic during sleep since discontinued breathing can awaken the sleeper. Accordingly, there is a need for apparatus and related methods for facilitating respiration during nasal congested sleep, particularly via preventing the tongue from forming a seal with the top of the mouth.

Various apparatus are known that may be used for preventing the blockage of the oral cavity during nasal congestion. For example U.S. Pat. No. 1,146,264 (issued Jul. 13, 1915), U.S. Pat. No. 2,521,039 (issued Sep. 5, 1950), U.S. Pat. No. 5,381,783 (issued Jan. 17, 1995), U.S. Pat. No. 5,427,117 (issued Jun. 27, 1995) and RE33,442 (issued Nov. 20, 1990) disclose a mouthpiece with an airway between the front teeth to allow breathing whenever the mouth-and-throat cavity would otherwise be sealed (e.g., during a procedure for setting a broken jaw or other event requiring clinched teeth (sports) or a particular jaw orientation (anti-snoring)). However, these apparatus have not adequately solved the above identified need since they do not necessarily prevent the interaction of the tongue with the palate whereby the oral cavity respiratory intake may still be obstructed or blocked. As a result, there is still a need for apparatus and related methods for facilitating respiration during nasal congested sleep, particularly via preventing the tongue from forming a seal with the palate.

Various apparatus are further known for controlling the behavior of a human tongue. For example: U.S. Pat. No. 2,037,079 (issued Apr. 14, 1936), U.S. Pat. No. 3,259,129 (issued Jul. 5, 1966), U.S. Pat. No. 3,871,370 (issued Mar. 18, 1975) and U.S. Pat. No. 3,295,519 (Jan. 3, 1967) disclose apparatus for preventing tongue thrust via poking, shocking, or blocking the tongue whenever the tongue is positioned at the front of the mouth behind the incisors (see also, e.g., U.S. Pat. No. 4,608,974 (issued Sep. 2 1986) which discloses a tongue obstructer that fits on the lingual side of a single tooth); U.S. Pat. No. 3,219,033 (issued Nov. 23, 1965) discloses a mouthpiece with a reservoir of syrup or sweets which cannot be accessed without certain tongue behavior; and, U.S. Pat. No. 4,169,473 (issued Oct. 2, 1979) and U.S. Pat. No. 4,304,227 (issued Dec. 8, 1981) disclose a mouthpiece featuring a socket for confining the tongue (see also, e.g., U.S. Pat. No. 5,092,346 (issued Mar. 3, 1992)). Yet, the disclosed devices for preventing tongue-thrust are aimed at preventing the tongue from interacting with the back of the teeth or forward palate and, even when worn, a respiratory seal may still be made between the tongue and the rear palate. Furthermore, a mouthpiece/retainer or syrup (candy) reservoir which conforms with the roof of the mouth is (1) not substantially different than the roof of the mouth with regards to sealing the respiratory intake via interaction with the tongue and (2) requires substantial customization. Mouthpieces with a tongue socket are also not adequately suited for alleviating blocked respiratory intake since fabrication is more complicated/expensive and since the device is bulky and uncomfortable whereby sleep remains difficult whether or not the airway is unobstructed. For these reasons, there is still a need for apparatus and related methods for comfortably facilitating respiration during nasal congested sleep, particularly via preventing the tongue from forming a seal with the palate.

Yet still, apparatus have been disclosed which depress the tongue to restrict its interaction with the palate to avoid respiration blockage without being undesirably bulky. For instance, U.S. Pat. No. 3,132,647 (issued Apr. 19, 1962), U.S. Pat. No. 3,312,216 (issued Jul. 22, 1963), and U.S. Pat. No. 5,052,409 (issued Oct. 1, 1991) disclose mouthpieces which are removably anchored to the upper teeth and molded with the palate whereby a springy-hinged arm may depress the tongue forward and increase the respiratory intake. However, such mouthpieces are also inadequate for solving the aforementioned needs since the construction of the springy-hinged arm is complicated and requires many moving parts. Furthermore, anchoring the mouthpiece to the upper teeth requires substantial customization with regard to the dimensions of the wearer's teeth/mouth. See, e.g., U.S. Pat. No. 5,052,409 col. 6:26-33. Additional drawbacks result from the placement of the hinged arm at the back of the tongue since, as taught by the U.S. Pat. No. 3,312,216, such placement is irritating and may actually cause the tongue to retract and thereby constrict the respiratory intake. See U.S. Pat. No. 3,312,216, col. 2:28-40. Therefore, a need still exists for apparatus and related methods for easily facilitating respiration during nasal congested sleep, particularly via comfortably preventing the tongue from forming a seal with the top of the mouth.

Additional mouthpiece designs are known which attempt to improve upon the mouthpiece designs heretofore disclosed. First, U.S. Pat. No. 7,007,697 (issued Mar. 7, 2006) teaches a mouthpiece with a bridge, anchored between the teeth and cheeks on both sides of the mouth, the bridge is extendable across the tongue to constrict tongue posture. The tongue bridge is not adequate for facilitating respiration since a tongue may nevertheless form a seal with the palate behind or in front of the bridge. Furthermore, the bridge may unseat from its anchor necessitating the presence of a safety handle to avoid swallowing by the wearer of the unseated mouthpiece. Second, U.S. Pat. No. 6,679,257 (issued Jan. 20, 2004) discloses a nozzle mouthpiece with a tongue depressor for providing a wearer with pressurized air. However, the mouthpiece is not suitable for use in nasal congested circumstances since the mouthpiece is not adapted for exhalation via the mouth cavity since the mouth is sealed to receive the pressurized air. Additionally, the tongue depressor is skinny whereby a restless tongue may avoid the restrictive function of the depressor via moving around the side thereof. Furthermore, the air passage of the mouthpiece terminates at the beginning of the tongue depressor rather than at the rear of the tongue depressor whereby the air is not provided to the wearer in closer proximity to the actual respiratory intake. While such a configuration may be adequate for forced air systems wherein the air is forced into the respiratory intake, such a system would not be adequate for non-forced air systems because the air could be confined to the front of the oral cavity. Accordingly, there is still a need for apparatus and related methods for easily facilitating respiration during nasal congested sleep, particularly via comfortably preventing the tongue from forming a seal with the palate.

In addition to the problems outlined above with regard to respiration, nasal congestion can also result in dry-out of the oral cavity, particularly where the cause of nasal congestion is bandages (as would be the case after nasal surgery). Dry out of the oral cavity is uncomfortable and can result in loss of sleep. For this reason there is a further need for an apparatus that facilitates salivation whereby the oral cavity does not dry out in the presence of bandages.

### SUMMARY OF THE INVENTION

It is an object of the present application to disclose apparatus and related methods for efficiently facilitating respiration during nasal congested sleep via comfortably preventing the tongue from forming a seal with the top of the mouth. In one non-limiting embodiment, a preferred apparatus is a mouthpiece which is received on a wearer's upper row of teeth. The mouthpiece suitably features a tongue depressor at a rear portion, a lip separator at a front portion, and at least one vent extending through the lip separator and the tongue depressor for delivery of air to the respiratory intake of the oral cavity. Further disclosed is a method of facilitating respiration comprising the step of: (1) positioning a mouthpiece between a wearer's teeth whereby a lip separator extends from the wearer's mouth and a tongue depressor forces the tongue away from the wearer's palate; and, (2) passing air through the lip separator and the tongue depressor, via the vent, to the proximity of the oral respiratory intake.

It is yet a further object of the present application to provide an apparatus for inducing salivation despite nasal congestion.

It is yet another object of the present application to meet the aforementioned needs without any of the drawbacks associated with apparatus heretofore known for the same purpose. It is yet still a further objective to meet these needs in an efficient and inexpensive manner.

### BRIEF DESCRIPTION OF THE FIGURES

The manner in which these objectives and other desirable characteristics can be obtained is better explained in the following description and attached figures in which:

FIG. 1 is a perspective view of an apparatus for facilitating respiration during nasal congestion.

FIG. 2 is an orthogonal front view of the apparatus of FIG. 1.

FIG. 3 is an orthogonal rear view of the apparatus of FIG. 1.

FIG. 4 is an orthogonal right-side view of the apparatus of FIG. 1.

FIG. 5 is an orthogonal left-side view of the apparatus of FIG. 1.

FIG. 6 is an orthogonal top view of the apparatus of FIG. 1

FIG. 7 is an orthogonal bottom view of the apparatus of FIG. 1

It is to be noted, however, that the appended figures illustrate only typical embodiments disclosed in this application, and therefore, are not to be considered limiting of its scope, for the invention may admit to other equally effective embodiments that will be appreciated by those reasonably skilled in the relevant arts. Also, figures are not necessarily made to scale.

### DETAILED DESCRIPTION OF PREFERRED EMBODIMENTS

In general, a preferred embodiment of the present application may be a mouthpiece 1 which, when worn during nasal congestion, depresses the tongue of a wearer and provides a delivery means for passing air to the oral respiratory intake. Suitably, the disclosed mouthpiece may be received in the vicinity of a wearer's upper row of teeth, the mouthpiece featuring a tongue depressor at a rear portion, a lip separator at a front portion, and at least one vent extending through the lip separator and the tongue depressor. Operably, the mouthpiece spreads the wearer's lips, depresses the wearer's tongue to prevent a seal from being created between the tongue and the palate, and provides an avenue or duct for air to pass into the mouth of the wearer to be received at the wearer's respiratory intake (i.e., the back of the throat). The more specific details and aspects of the disclosed embodiment are further described below with reference to the figures.

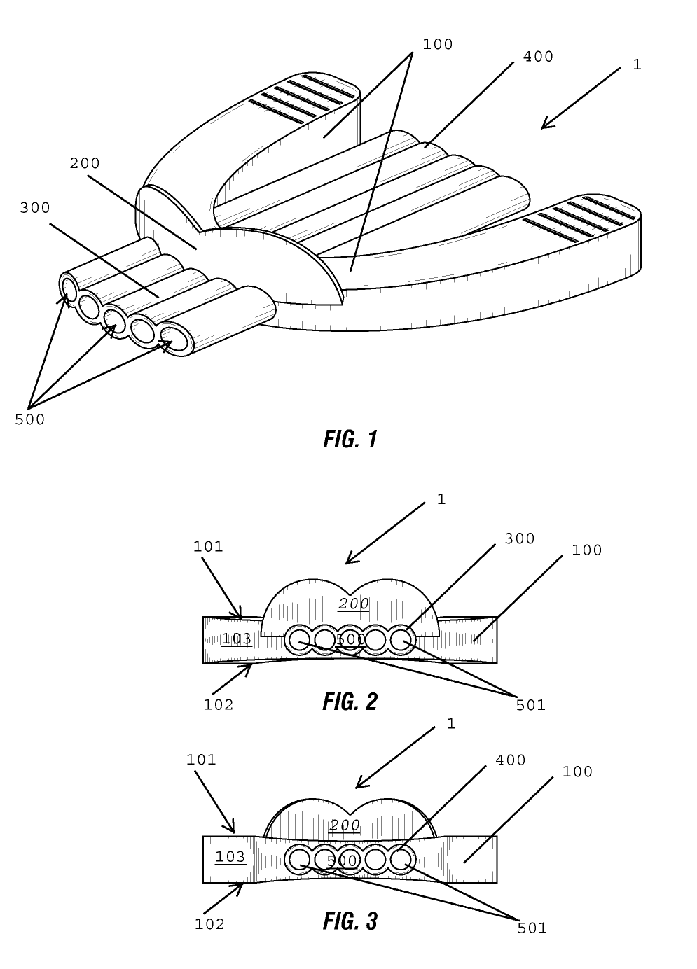

FIG. 1 is a perspective view of a preferable mouthpiece 1. As seen in the figure, the mouthpiece 1 may comprise: a teeth-seat 100; an anchor 200; a lip-separator 300; a tongue depressor 400; and, an air vent 500 disposed through the lip separator 300, the teeth-seat 100, and the tongue depressor 400. FIGS. 2 through 7 depict the six orthogonal views of the mouthpiece 1 depicted in FIG. 1. More specifically, FIGS. 2 through 7 respectively depict the front, rear, right-side, left-side, top, and bottom views of the mouthpiece 1 depicted in FIG. 1. Taken together, FIGS. 1 through 7 suitably illustrate the above referenced components of the depicted mouthpiece 1.

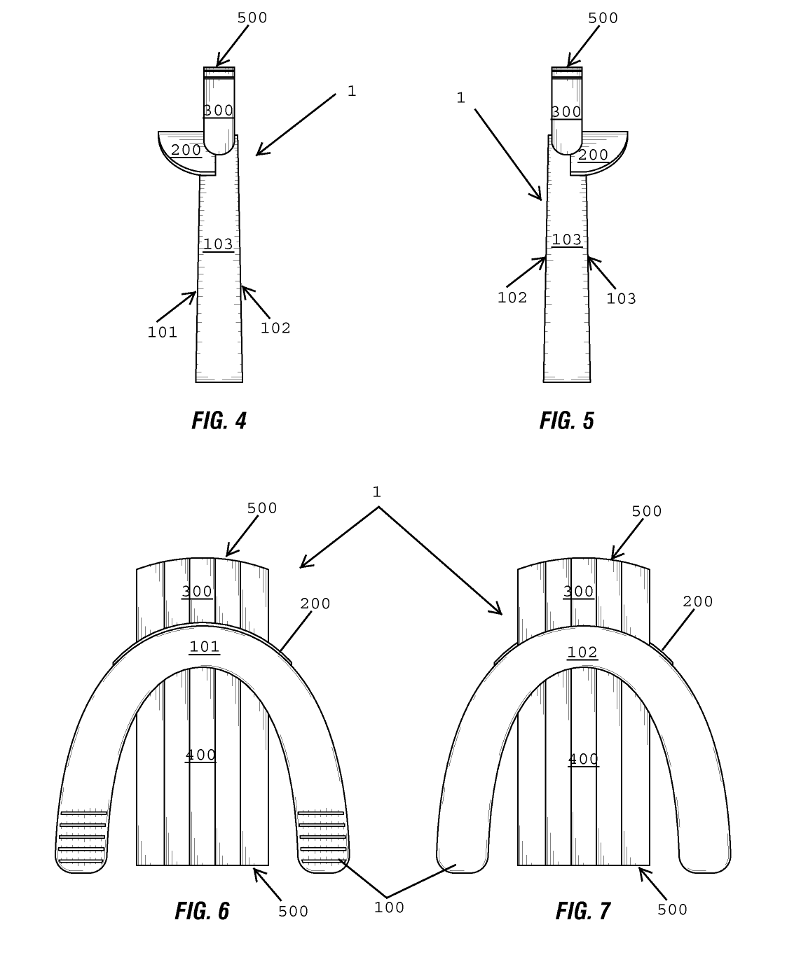

The teeth-seat 100 is best illustrated in FIGS. 1 and 4 through 7. Referring to these figures, the teeth-seat 100 is typically horse-shoe shaped (U-shaped) to generally match the typical plan of the upper row of human teeth. Operably, the teeth-seat 100 suitably features: an upper surface 101 for receiving the wearer's upper row of teeth (see FIG. 6); a lower surface 102 for interacting with the wearer's lower row of teeth (see FIG. 7); and a thickness 103 for comfortably spacing the wearer's teeth whereby the mouth is propped ajar for allowing air intake (see FIGS. 4 and 5). The upper surface 101 of the teeth-seat 100 may be optionally molded to fit a wearer's teeth for increased comfort and customized application of the mouthpiece 1.

The anchor 200 is best seen in FIGS. 1 through 5. Referring to these figures, the anchor 200 defines an upward projection at the forefront of the teeth-seat 100 for insertion between the upper lip and teeth of a wearer (i.e., the wearer's vestibule) (see FIGS. 2 and 3). As suggested by the term “anchor,” the anchor 200 aids in retaining the mouthpiece at the upper portion of the wearer's mouth with the wearer's teeth-seated in the teeth-seat 100 as identified above. As with the teeth-seat 100, the anchor 200 may be molded for a customized fit between a wearer's upper lip and teeth to provide greater retentive properties therein and the disclosed embodiment is only one of many shapes which could serve the purpose.

The lip separator 300 is best depicted in FIGS. 1, 2 and 4 through 7. The lip-separator 300 preferably defines a forward projection centrally positioned at the forefront of the teeth-seat 100 between the upper 101 and lower 102 surfaces (see FIGS. 2, 6, and 7). Operably, the lip-separator 300 extends through a mouthpiece 1 wearer's upper and lower lips whereby the lips are propped ajar for allowing air intake. Unlike the teeth-seat 100 and the anchor 200, the lip separator 300 should preferably not be molded for a customized fit with the users mouth for reasons disclosed below. Various shapes may serve the lip-separator 300 function, however, an air passageway, as discussed further below, extends therethrough and may also take various shapes.

The tongue depressor 400 is most easily viewed in FIGS. 1, 3, 6, and 7. The tongue depressor 400 suitably defines a rearward projection which is centrally positioned within the general horse-shoe shape of the teeth-seat 100 whereby the depressor 400 occupies the area between the wearer's upper teeth row, when worn. Preferably, the tongue depressor 400, when worn, generally extends centrally over the wearer's tongue from behind the wearer's incisors to within the oral cavity at a distance that is approximately equidistant to the inner most molar of the wearer. Operably, the tongue depressor 400 preferably occupies the area between the wearer's palate and tongue in order to confine the tongue's posture to the lower portion of the oral cavity and thereby forestall the creation of a seal between the tongue and the palate. The tongue depressor 400 should preferably be wide enough to prevent the movement of the tongue into the upper portions of the oral cavity via circumvention of the tongue depressor 400. Suitably, the depressor 400 may be truncated or extended for customizing the comfort (e.g., gag) and tongue depressive properties of the tongue depressor 400. In a preferable embodiment, the tongue depressor 400 is suitably fabricated for deep penetration into the mouth of a wearer but truncatable at the election of the user. A suitable mode of truncation may be cutting the depressor 400 with a knife or scissor. Other truncation modes will be known to one skilled in the art.

Another function of the tongue depressor 400 may be to cause or normalize salivation. Interaction of the depressor 400 with the wearer's tongue naturally enhances the normalized and/or additional saliva. The additional or normal quantity of saliva is preferably allowed to naturally circulate within the wearer's mouth to prevent dry-out of the oral cavity.

The suitable details of the vent 500 are best depicted by FIGS. 1 through 3, 6, and 7. The vent 500 is preferably a delivery means for either: (1) passing air to the respiratory intake within the oral cavity: or, (2) exhausting respiratory products from the respiratory outlet of the oral cavity. Generally, the vent 500 defines a plurality of channels 501 through the mouthpiece 1 whereby the respiratory outlet of the oral cavity and the ambient atmosphere are in fluid communication. More specifically, the vent 500 defines at least one channel (five channels depicted in the drawings) that extends from the distal portion of the lip separator 300, through the teeth-seat 100, to the distal portion of the tongue depressor 400 whereby air may be moved to within the mouth, or respiratory exhaust discharged therefrom, despite the retention of the mouthpiece 1 within the wearer's mouth.

It should be noted that the channels define a duct for fluid communication between the respiratory intake/exhaust of the oral cavity and the ambient atmosphere. Although the channels 501 depicted in FIGS. 1 through 7 are circularly tubiform, the channel(s) may be any configuration that results in the stated fluid communication. Other channel configurations may become readily apparent to one skilled in the art once this disclosure is consulted.

As alluded to above, the teeth-seat 100 and anchor 200 may be molded for customized retention of the mouthpiece within the wearer's oral cavity. Typically, molding of the apparatus may be accomplished via boiling the mouthpiece 1 until it is soft so that the wearer may bite down on the teeth-seat 100 whereby the mouthpiece deforms and removably re-solidifies around the wearer's teeth and vestibule. However, because the vent 500 is disposed through the teeth-seat 100 it is preferable that the vent not be kinked during the molding process. Kinking of the vent 500 may be avoided by, among other ways, placing a removable support into the channels 501 during the molding process and then subsequently removing the support after re-solidification. A suitable material for construction of the mouthpiece 1 is ethelen-vinyl acetate which will soften for molded customization under temperatures associated with boiling water and re-solidify at room temperature. Alternatively, impressions of the wearer's teeth may be incorporated into the original casting of the mouthpiece 1. Casting of the mouthpiece may suitably be accomplished via blow or injection molding processes or their equivalent. Those skilled in the art will know well the mechanisms available for fabricating the mouthpiece 1 and/or molding the disclosed mouthpiece 1 around a wearer's teeth.

The above disclosed apparatus provides distinct advantages over the state of the prior art. Among other things, the disclosed apparatus: restricts interaction between the palate and the tongue without complicated or bulky tongue suppression components or mechanisms; provides a delivery means for passing air to a close proximity from the oral respiratory intake; provides a delivery means for passing exhaust from a close proximity from the oral respiratory outlet; and, features a truncatable depressor for customizing the apparatus for enhanced performance and user comfort. As set forth in the background above, none of these advantages are found in the prior art

It should be noted that FIGS. 1 through 7 and the associated description are of illustrative importance only. In other words, the depiction and descriptions of the present invention should not be construed as limiting of the subject matter in this application. For example, the teeth-seet 100 may be divided vertically to provide separated upper and lower recepticals for the wearer's upper and lower rows of teeth respectively. Additional modifications may become apparent to one skilled in the art after reading this disclosure.

I claim:

1. A mouthpiece comprising:
a U-shaped teeth-seat, said teeth-seat operably configured to receive the upper teeth of a wearer and prop ajar the mouth of the wearer;
a lip separator forwardly projecting from the teeth-seat, said lip separator operably configured to prop ajar the lips of a wearer;
a tongue depressor rearwardly projecting centrally with regard to the U-shaped teeth-seat, said tongue depressor operably configured to confine the tongue thereunder whereby the tongue and palate cannot seal the respiratory intake of the wearer's oral cavity; and,
a vent defined by at least one channel through the lip separator, teeth-seat, and tongue depressor whereby the oral cavity and ambient atmosphere are in fluid communication therethrough, said channel operably configured to pass air or respiratory exhaust.

2. The mouthpiece of claim 1 further comprising an anchor.

3. The mouth piece of claim 2 wherein the anchor and teeth-seat are molded to fit a human's vestibule and teeth respectively.

4. The mouth piece of claim 1 wherein the mouthpiece is constructed of ethylene-vinyl acetate.

5. The mouth piece of claim 1 wherein the tongue depressor is truncatable.

6. The mouth piece of claim 1 wherein the channel(s) is tubiform

7. A method of facilitating the respiration of a human in circumstances wherein the human suffers from nasal congestion, the method comprising the step of:
obtaining a mouthpiece that is operably configured, when worn, to depress the tongue of a wearer, said mouth piece comprising a vent means extending therethrough said mouthpiece for passing or air to the oral respiratory intake; and,
passing air to the rear portion of the human's oral cavity via the vent means.

8. The method of claim 7 wherein the mouth piece and vent means are defined by:
a U-shaped teeth-seat;
a lip separator forwardly projecting from the teeth-seat;
a tongue depressor rearwardly projecting centrally to the U-shaped teeth-seat; and,
a vent defined by at least one channel through the lip separator, teeth-seat, and tongue depressor whereby the oral cavity and ambient atmosphere are in fluid communication therethrough, said channel operably configured to pass air or respiratory exhaust.

9. The method of claim 8 further comprising the steps of:
propping ajar the human's teeth via the teeth-seat;
propping open the human's lips via the lip separator;
confining the tongue of the human beneath the tongue depressor; and,
passing air to the rear portion of the human's oral cavity via the channel(s).

10. The method of claim 8 wherein the mouthpiece further comprises an anchor.

11. The method of claim 9 further comprising the step of molding the anchor and teeth-seat to custom fit the human's vestibule and teeth respectively.

12. The method of claim 8 wherein the mouthpiece is constructed of ethylene-vinyl acetate.

13. The method of claim 8 wherein the tongue depressor is truncatable.

14. The method of claim 12 further comprising the step of truncating the tongue depressor according to the human's preferences.

15. The method of claim 8 wherein the channel(s) is tubiform

16. A mouthpiece operably configured, when worn, to depress the tongue of a wearer, said mouth piece comprising a vent means extending therethrough said mouthpiece for passing or air to the oral respiratory intake.

17. The mouth piece of claim 16 further comprising:
a U-shaped teeth-seat;
a lip separator;
a tongue depressor; and,
wherein the vent means extends through the lip separator, teeth-seat, and tongue depressor.

18. The mouthpiece of claim 17 further comprising an anchor.

19. The mouth piece of claim 18 wherein the anchor and teeth-seat are moldable to fit a human's vestibule and teeth respectively.

20. The mouth piece of claim 19 wherein the tongue depressor is truncatable.
